(19) United States
(12) Patent Application Publication
(10) Pub. No.: US 2020/0281956 A1
(43) Pub. Date: Sep. 10, 2020

(54) ANTIBACTERIAL COMPOSITION CONTAINING A DEOXYHEXOSE ALKYL MONOACETAL OR MONOETHER

(71) Applicants: TEREOS STARCH & SWEETENERS BELGIUM; UNIVERSITÉ CLAUDE BERNARD LYON 1; CENTRE NATIONAL DE LA RECHERCHE SCIENTIFIQUE

(72) Inventors: Charlotte GOZLAN, Villeurbanne (FR); Dorine BELMESSIERI, Villeurbanne (FR); Marie-Christine DUCLOS, Villeurbanne (FR); Nicolas DUGUET, Villeurbanne (FR); Marc LEMAIRE, Villeurbanne (FR); Gérard LINA, Villeurbanne (FR); Oana DUMITRESCU, Villeurbanne (FR); Andreas REDL, Aalst (BE)

(21) Appl. No.: 16/063,679

(22) PCT Filed: Dec. 19, 2016
(86) PCT No.: PCT/IB2016/057780

§ 371 Date: Jun. 18, 2018

(30) Foreign Application Priority Data
Dec. 17, 2015 (FR) 15/02629

(51) Int. Cl. A61K 31/7028; A61P 31/04

(52) U.S. Cl. CPC A61K 31/7028; A61P 31/04

(57) ABSTRACT

The present invention relates to a A bactericidal or bacteriostatic composition comprising a deoxyhexose alkyl acetal or ether or the mixture of isomers thereof, the use thereof in treating or preventing Gram-positive bacterial infections, the use thereof as hygiene or dermatological product for external use, and also a method for disinfecting surfaces.

The present application claims priority from French Patent Application No. 15/02629 filed on Dec. 17, 2015, which is incorporated herein by reference.

### TECHNICAL FIELD

The present disclosure relates to a bactericidal or bacteriostatic composition comprising an alkyl acetal or alkyl ether of deoxyhexose or of a deoxyhexose derivative, in which the alkyl group comprises between 11 and 18 carbon atoms, a pharmaceutically acceptable salt, an isomer or a mixture of isomers thereof, the use thereof in treating or preventing Gram-positive bacterial infections, the use thereof as hygiene or dermatological product for external use, and also a method for disinfecting surfaces.

### TECHNICAL BACKGROUND

Antimicrobial compounds are defined as molecules capable of inhibiting or stopping the growth of microorganisms, or of killing them. In this context, they are commonly used to prevent or treat human and animal infections, and in the food-processing industry to prevent multiplication of pathogenic bacteria in food. Widespread use of antimicrobial compounds has promoted the emergence of resistant infectious agents. The spread of bacteria that has acquired resistance mechanisms for the most widely used antimicrobial compounds is an increasingly alarming major public health problem (J. S. Bradley et al. Lancet Infect. Dis. 2007; 7:68-78).

As an illustration, numerous strains resistant to antibiotics for the most pathogenic species of genus Staphylococcus, namely Staphylococcus aureus, have been isolated. Staphylococcus infections represent a high percentage of serious infections. What is more, almost half of nosocomial infections are reportedly related to staphylococcus. Mention may also be made of the numerous strains of Enterococcus faecalis or Enterococcus faecium that are resistant to commonly used antibiotics. Although they are less virulent than staphylococci especially, an increasing number of multiresistant enterococcus strains and more recently epidemics of enterococci resistant to glycopeptides, the antibiotics of recourse for this bacterial family, have been identified.

Another phenomenon of antibiotic resistance has been described that might not only be related to the excessive use of antibiotics, but to food preservation methods. Thus, for example, it has been shown that Listeria monocytogenes is more resistant to antibiotics after having survived osmotic stress, at a low temperature or in an acidic medium (Anas A. et al. (2015) Food Microbiology, Volume 46, April, Pages 154-160). Yet, the human contamination comes from food. In addition, although it is relatively rare, human listeriosis is a serious infection with mortality estimated at 50%. Accordingly, the emergence of antibiotic resistance in L. monocytogenes that could be caused by modern food preservation or treatment methods constitutes a serious threat to public health.

Although several mechanisms are often simultaneously involved in antibiotic resistance, it is common to classify them into three categories: (a) defective penetration of the antibiotic into the bacterium, (b) inactivation or excretion of the antibiotic by bacterial enzymatic systems and (c) lack of affinity between bacterial target and antibiotic. These three categories of resistance mechanisms have a structural component, i.e. the mechanisms used are dependent on the structure of the molecule in question.

No process in the prior art makes it possible to produce an isomeric mixture of biobased compounds with low toxicity and at low cost.

Nevertheless, biobased compounds have been described by the prior art. Thus, the prior art describes different compounds used as antimicrobials, among which are fatty acids and their corresponding polyhydroxylated esters that are active against Gram-positive bacteria and having long aliphatic chains. By way of indication, one of the most active antimicrobials is monolaurin, a glycerol monoester having a C12 aliphatic chain. Its trade name is LAURICIDIN®. This compound is used as a food additive with the aim of inhibiting bacterial growth (E. Freese, C. W. Sheu, E. Galliers. Nature 1973, 241, 321-325; E. G. A. Verhaegh, D. L. Marshall, D.-H. Oh. Int. J. Food Microbiol. 1996, 29, 403-410). However, since the ester function of the monolaurin is sensitive to esterases, this compound is quickly degraded and has a poor half-life.

The prior art also describes antimicrobials derived from sugar considered to be particularly attractive because of their biodegradability, their low toxicity and environmental impact.

Examples of antimicrobials derived from sugar are the esters derived from sugar that are also used industrially for antimicrobial applications because their raw materials and production costs remain relatively low. Mention may be made, for example, of sorbitan caprylate described in international patent application WO 2014/025413 as a mixture with Hinokitiol in an antimicrobial formulation. According to this application, this formulation allegedly makes it possible to inhibit or kill Gram-positive and Gram-negative bacteria, fungi and/or yeast.

The prior art also describes the use of disaccharide esters as antimicrobial agents in the food industry. Dodecanoyl sucrose is one of the most commonly used. The latter is allegedly particularly active against L. monocytogenes (M. Ferrer, I Soliveri, F. J. Plou, N López-Cortes, D. Reyes-Duarte, M Christensen, J. L. Copa-Patiño, A. Ballesteros, Enz. Microb. Tech., 2005, 36, 391-398). Nonetheless, it is also described as weakly inhibiting the growth of S. aureus, for applications in hospitals (J. D. Monk, L. R. Beuchat, A. K. Hathcox, J. Appl. Microbial. 1996, 81, 7-18). Thus, the sucrose ester is reported to have properties that are bacteriostatic (stopping bacterial growth) but not bactericidal (killing the bacteria).

In addition, the synthesis of sugar esters presents numerous drawbacks. First, in spite of the low production cost, synthesizing esters, more particularly for di- and trisaccharides, is problematic because of the high functionality of sugars, which causes the formation of a mixture of mono-, di- and polyesters and the presence of a polar solvent, such as dimethylformamide (DMF) and pyridine, is generally necessary to better solubilize the highly polar reagents. However, these solvents are classed carcinogenic, mutagenic and reprotoxic (CMR) and their use must be avoided. To solve this problem, enzymatic synthesis was used but the need to work with very dilute media in these conditions makes production limited.

Moreover, the ester functions of these compounds are readily hydrolysable by the esterases present in the cells. However, the molecules released after this hydrolysis, i.e. the sugar and the fatty acid, have little or no antimicrobial properties (the fatty acid is slightly active). This causes instability that is responsible for a reduced activity time of these compounds.

### DETAILED DESCRIPTION

### Bactericidal or Bacteriostatic Composition

In order to obtain an antibiotic composition having lower chances of allowing resistance to develop, the inventors have envisaged the use of a composition containing a mixture of compounds having antibiotic activity but comprising minor structural differences capable of reducing the chances of developing bacterial resistance. Thus, they have envisaged a composition comprising an isomeric mixture of compounds having antibiotic activity.

The inventors wished to develop an antibiotic composition also having low toxicity and low environmental impact; a biodegradable antibiotic composition that can be obtained in large quantities from renewable resources, at low cost so as to be perfectly accessible for industrial application but also as effective as non-biobased antimicrobials.

Thus, in order to produce an antibiotic composition that is not prone to developing resistance, comprising effective and stable antimicrobial agents, the invention proposes in some embodiments a deoxyhexose alkyl monoacetal or monoether, in which the alkyl group comprises between 11 and 18 carbon atoms, preferentially in the form of a mixture of regioisomers and/or diastereoisomers obtained under inexpensive conditions while respecting the environment and not representing a hazard for topical applications or applications by ingestion.

The invention in some embodimnts relates to a bactericidal or bacteriostatic composition, characterized in that it comprises a deoxyhexose alkyl ether or alkyl acetal and/or a glycosylated and/or hydrogenated and/or dehydrated deoxyhexose alkyl ether or alkyl acetal, in which the alkyl group comprises between 11 and 18 carbon atoms, a pharmaceutically acceptable salt, an isomer or a mixture of isomers thereof; preferentially, said alkyl ether or alkyl acetal radical is in the 2-O—, 3-O—, or 4-O— position, the isomers preferentially being chosen from regioisomers and/or diastereoisomers. Typically, the deoxyhexose is chosen from rhamnose or fucose. Advantageously, the deoxyhexose alkyl ether or alkyl acetal is a deoxyhexose alkyl monoacetal or monoether. Typically, said deoxyhexose is a glycosylated and/or hydrogenated and/or dehydrated deoxyhexose.

The invention also relates in some embodiments to a composition comprising an alkyl monoacetal or monoether of deoxyhexose or of a deoxyhexose derivative or a mixture of isomers thereof, said deoxyhexose derivative being a glycosylated and/or hydrogenated and/or dehydrated deoxyhexose, the isomers being preferentially chosen from regioisomers and/or diastereoisomers, said alkyl monoacetal or monoether of deoxyhexose or of a deoxyhexose derivative or the mixture of isomers thereof being obtained by a process comprising the following steps:
a) acetalization or trans-acetalization of a deoxyhexose or a deoxyhexose derivative by an aliphatic aldehyde containing from 11 to 18 carbon atoms or the acetal thereof
b) optional catalytic hydrogenolysis of the alkyl acetal of deoxyhexose or of the deoxyhexose derivative obtained in a) preferentially without acid catalyst, and
c) recovery of a mixture of isomers of alkyl monoethers of deoxyhexose or of the deoxyhexose derivative obtained in b), in which the alkyl group (R) comprises between 11 and 18 carbon atoms or
   recovery of a mixture of isomers of alkyl monoacetals of deoxyhexose or deoxyhexose derivative obtained in a), in which the alkyl group (R) comprises between 11 and 18 carbon atoms.

A “deoxyhexose” must be understood to be a hexose in which one of the hydroxyl groups (OH) has been replaced by a hydrogen. The deoxyhexoses may be isolated from plants; for example, rhamnose is derived from buckthorn (Rhamnus) or sumac, and fucose from membrane polysaccharides from mammal or insect cells. An example of a suitable deoxyhexose may be fucose or rhamnose.

As used here, the term “rhamnose” refers to D-(−)-rhamnose or to L-(+)-rhamnose. Also referred to as isodulcitol or 6-deoxy-L-mannose, rhamnose is a hexose of empirical formula C₆H₁₂O₅.

As used here, the term “fucose” refers to D-(−)-fucose or to L-(+)-fucose. Also referred to as 6-deoxy-L-galactose, fucose is a hexose of empirical formula C₆H₁₂O₅.

According to one embodiment, the deoxyhexose is a glycosylated and/or hydrogenated and/or dehydrated deoxyhexose. Such glycosylated and/or hydrogenated and/or dehydrated deoxyhexoses are termed “deoxyhexose derivatives” in the present document. For example, the rhamnose derivative is an anhydrorhamnose or rhamnitol.

An “anhydrorhamnose” must be understood to be a compound obtained by dehydration, by the elimination of one or more molecules of water from the rhamnose. An example of an anhydrorhamnose may be 1,2-anhydrorhamnose, 1,2-anhydrorhamnose, 1,3-anhydrorhamnose or 2,3-anhydrorhamnose.

The anhydrorhamnose may be obtained by the dehydration of rhamnitol to form, for example, 1,5-anhydrorhamnitol.

According to one embodiment, said rhamnose derivative is a sugar alcohol, rhamnitol. As it is used here, the term “sugar alcohol”, also referred to as “polyol”, refers to a hydrogenated monosaccharide form in which the carbonyl group (aldehyde or ketone) has been reduced to a primary or secondary hydroxyl.

Similarly, when the deoxyhexose is a fucose, said hydrogenated fucose derivative is a sugar alcohol, fucositol.

Advantageously, the fucose derivative is an anyhydrofucose. An “anhydrofucose” must be understood to be a fucose obtained by dehydration, by the elimination of one or more molecules of water from the fucose. An example of an anhydrofucose may be 1,2-anhydrofucose or 1,3-anhydrofucose.

According to one embodiment, the process according to the invention may comprise a step of dehydration of said deoxyhexose or of the derivative thereof, in order to obtain, for example, a monoanhydrorhamnose or a monoanhydrofucose. The deoxyhexose is typically melted before the dehydration step. The dehydration step may be carried out with a catalyst, for example with an acid catalyst.

According to some embodiments of the invention, the dehydration step is carried out under a hydrogen atmosphere with a pressure preferably of approximately 20 to 50 bar.

Advantageously, the dehydration step is carried out at a temperature of between 120 and 170° C., preferably between 130 and 140° C.

The deoxyhexose is typically purified after the dehydration step, for example by crystallization, recrystallization or chromatography. According to one embodiment, said deoxyhexose derivative is a glycosylated deoxyhexose, in other words an alkyl glycoside.

As used here, the term “glycosylated” or “glycosylation” refers to a reaction between an alkyl group and a saccharide at the anomeric position (hemiacetal function) of the saccharide, to give rise to a mixed acetal function (IUPAC Compendium of Chemical Terminology Gold book Version 2.3.3 2014-02-24 p. 635-636 and PAC, 1995, 67, 1307 (“Glossary of class names of organic compounds and reactivity intermediates based on structure” IUPAC Recommendations 1995) page 1338 White Book, p. 136). This glycosylation reaction is in opposition to alkylation in that the latter may take place between an alkyl group and a saccharide but on any oxygen of the saccharide, to form an ether function.

As used here, the term “alkyl glycoside” refers to a deoxyhexose in which the reducing part is connected via a bond to an alkyl group by glycosylation, as described in the prior art. Typically, the deoxyhexose or the derivative thereof may be connected to the alkyl group via an oxygen atom (an O-glycoside), a nitrogen atom (a glycosylamine), a sulfur atom (a thioglycoside), or a carbon atom (a C-glycoside). The alkyl group may have a varied chain length; preferably, the alkyl group is a C1-C4 alkyl group. A yet further preferred alkyl group is a methyl or an ethyl. Typically, the glycosylated deoxyhexose is a glycosylated rhamnose, a glycosylated rhamnitol, a glycosylated fucose or a glycosylated fucositol. Alkyl glycosides may for example be selected from a group consisting of methyl rhamnoside, ethyl rhamnoside, propyl rhamnoside, butyl rhamnoside, methyl fucoside, ethyl fucoside, propyl fucoside and butyl fucoside.

According to some embodiments of the invention, the step of acetalization or trans-acetalization comprises:

i) an optional step of preheating said deoxyhexose or derivative thereof, preferably to a temperature of between 70 and 130° C., typically between 90 and 110° C.,

ii) a step of addition of the aliphatic aldehyde or of a deoxyhexose aliphatic aldehyde derivative or to the derivative thereof, and

iii) a step of addition of a catalyst, preferably of an acid catalyst.

Typically, the acetal of an aliphatic aldehyde may be a dialkyl acetal of the corresponding aldehyde. Dimethyl acetals and diethyl acetals are preferred.

Step i) is particularly advantageous in that it may be carried out in the absence of solvent.

Preferably, the acid catalyst used during the step of acetalization or of trans-acetalization and, where appropriate, during the step of dehydration, may be a homogeneous or heterogeneous acid catalyst. The term “homogeneous” as used in the expression “homogeneous acid catalyst” refers to a catalyst which is in the same phase (solid, liquid or gas) or in the same aggregate state as the reagent. Conversely, the term “heterogeneous” as used in the expression “heterogeneous acid catalyst” refers to a catalyst which is in a different phase (solid, liquid or gas) than the reagents.

Said acid catalyst used during the step of acetalization or of trans-acetalization and, where appropriate, during the step of dehydration, may be independently selected from solid or liquid, organic or inorganic acids, solid acids being preferred. In particular, the preferred acid catalyst is chosen from para-toluenesulfonic acid, methanesulfonic acid, camphosulfonic acid (CSA) and sulfonic resins.

Typically, the step of acetalization or of trans-acetalization is carried out at temperatures between 70 and 130° C., typically between 70 and 90° C. The temperature of the reaction mixtures may vary as a function of the reagents and solvents used. The reaction time is determined by the degree of conversion achieved.

According to one embodiment, the step of acetalization or of trans-acetalization may be carried out by an aliphatic aldehyde or the acetal thereof, typically, a linear or branched aliphatic aldehyde or the acetal thereof. The step of acetalization or of trans-acetalization may typically be carried out with an aliphatic aldehyde or the acetal thereof having 11, 12, 13, 14, 15, 16, 17 or 18 carbon atoms, for example chosen from undecanal, dodecanal, tridecanal, tetradecanal, pentadecanal, hexadecanal, heptadecanal, octadecanal and acetal. Preferably, the C11-C13 aliphatic aldehyde or the acetal thereof is a C12 aliphatic aldehyde or the acetal thereof, for example a dodecanal or the acetal thereof.

The expression “the acetal thereof” or “the acetals thereof” as used in the present text encompasses the dialkyl acetal of the corresponding C11-C18 aliphatic aldehyde. More particularly, dimethyl or diethyl acetals of the C11-C18 aliphatic aldehyde are preferred.

According to one embodiment, the step of acetalization or of trans-acetalization may be carried out with or without solvent. When the reaction is carried out in the presence of a solvent, the solvent is preferably a polar solvent.

Typically, the solvent may be chosen from dimethylformamide (DMF), dimethyl sulfoxide (DMSO), dimethylacetamide (DMA), acetonitrile (CH₃CN), tetrahydrofuran (THF), 2-methyl tetrahydrofuran (2Me-THF), cyclopentyl methyl ether (CPME), methanol (MeOH), ethanol (EtOH), propanol (PrOH), isopropanol (iPrOH), butanol (BuOH), dibutyl ether (DBE), methyl tert-butyl ether (MTBE) and trimethoxypropane (TMP). In-depth experimental studies have led to the selection of conditions enabling optimal yields and degrees of conversion to be observed during the steps of acetalization or of trans-acetalization. The best results were obtained when the molar ratio of [(C11-C18 aliphatic aldehyde or acetal thereof):deoxyhexose or derivative thereof] is between 5:1 and 1:5, preferably between 4:1 and 1:4, and advantageously between 3:1 and 1:3.

The inventors have more particularly shown that, during an acetalization reaction, the molar ratio of C11-C18 aliphatic aldehyde:(deoxyhexose or derivative thereof) of between 1:1 and 1:5, preferably between 1:1 and 1:4, and preferably between 1:3 and 1:2 improves the yields and provides optimal degrees of conversion.

The inventors have also shown that, during trans-acetalization reactions, a molar ratio of C11-C18 aliphatic acetal : (deoxyhexose or derivative thereof) of between 1:1 and 5:1, preferably between 5:4 and 4:1, preferably between 3:1 and 4:3, more preferably still between 3:2 and 2:5 improves the yields and provides optimal degrees of conversion. The catalysts used are the same as during the acetalization reaction.

According to one embodiment, the process of the invention also comprises at least one step of neutralization and/or filtration and/or purification after any one of the steps of dehydration where appropriate, of acetalization or of trans-acetalization.

When a step of purification is provided, said purification step may for example be a crystallization, a recrystallization or chromatography. The chromatography is preferably carried out using a non-aqueous polar solvent. In general, when a step of filtration and/or purification is provided before the step of hydrogenolysis, the non-aqueous polar solvent may be identical to that used during the step of hydrogenolysis.

Advantageously, the step of hydrogenolysis is carried out at a temperature of between 80° C. and 140° C., and/or at a hydrogen pressure of between 15 and 50 bar, preferably between 20 and 40 bar.

The step of hydrogenolysis is advantageously carried out in an aprotic polar solvent, preferably a non-aqueous solvent. This is because aprotic solvents afford better conversion. Examples of aprotic solvents are, inter alia and without limitation, alkanes, 1,2,3-trimethoxypropane (TMP), methyl tert-butyl ether (MTBE), tetrahydrofuran (THF), 2-methyl tetrahydrofuran (2Me-THF), dibutyl ether (DBE) and cyclopentyl methyl ether (CPME). The aprotic solvent is preferably CPME. Alkanes are advantageous since they enable better solubilization of the hydrogen in the medium. However, the conversion is lower than with other aprotic solvents such as CPME. In general, among the alkanes, preference is given to dodecane and heptane.

The step of hydrogenolysis is preferably carried out in a polar aprotic solvent at a temperature of between 80° C. and 140° C. and/or under a hydrogen pressure of between 15 and 50 bar, in the presence of a catalyst suited to hydrogenolysis reactions.

Preferably, the step of hydrogenolysis is carried out in a non-aqueous polar solvent at a temperature of between 100° C. and 130° C. and/or at a pressure of between 25 and 35 bar.

Generally, the hydrogenolysis is carried out in the presence of a suitable catalyst such as a catalyst based on precious metals or common metals. More particularly, the common metals may be ferrous or non-ferrous metals. Typically, the hydrogenolysis is carried out in the presence of a catalyst based on ferrous metals.

By way of indication, a metal catalyst belonging to the group of the ferrous metals may be nickel, cobalt or iron.

Preferably, the hydrogenolysis is carried out using a catalyst based on precious metals such as palladium, rhodium, ruthenium, platinum or iridium.

As a general rule, the catalyst used during hydrogenolysis may be fixed on a support such as carbon, alumina, zirconia or silica or any mixture thereof. Such a support is for example a bead. Thus, a palladium catalyst fixed on carbon beads (Pd/C) may be advantageously used. These catalysts may be doped by adding precious metals or common metals. These are called doping agents. Typically, the doping agent represents 1 to 10% by weight of the catalyst.

The invention in some embodiments also relates to a bactericidal or bacteriostatic composition comprising a mixture of positional isomers of deoxyhexose alkyl monoacetals or monoethers having an alkyl ether or alkyl acetal radical on 2 distinct positions of the deoxyhexose or of the deoxyhexose derivative, and also the pharmaceutically acceptable salts thereof, in which the alkyl group comprises between 11 and 18 carbon atoms, preferentially from 11 to 13 carbon atoms.

The term “pharmaceutically acceptable salts” denotes any salt which, by administering to the patient, is capable of (directly or indirectly) providing a compound such as that described presently. The salts may be prepared by processes known in the prior art.

“Isomers” is intended to mean molecular entities which have the same atomic composition (molecular formula) but different linear formulae or different stereochemical formulae (PAC, 1994, 66, 1077 (Glossary of terms used in physical organic chemistry (IUPAC Recommendations 1994)) page 1129.

Typically, the isomers are regioisomers and/or diastereoisomers.

According to the present invention, the term “diastereoisomer” refers to stereoisomers (isomers which have an identical constitution but which differ in the spatial arrangement of their atoms) which are not enantiomers (molecular entities having stereochemical formulae which form non-superimposable mirror images).

According to the present invention, the term “regioisomer” or the expression “positional isomer” refers to isomers in which a functional group is placed on different carbons of the carbon-based chain. More particularly, this is intended to mean isomers of alkyl monoacetals or monoethers of deoxyhexose or of a deoxyhexose derivative, in which the alkyl monoacetal or monoether radical is positioned on different oxygens present on the backbone of the deoxyhexose or of the deoxyhexose derivative.

Typically, mention may be made, for example, of methyl 2,3-O-dodecylidene α-L-rhamnopyranoside and/or methyl 2-O-dodecyl α-L-rhamnopyranoside and/or methyl 3-O-dodecyl α-L-rhamnopyranoside.

Typically, the composition is bactericidal or bacteriostatic with respect to Gram-positive bacteria. The invention in some embodiments also relates to the use of such a composition as agent that is bactericidal or bacteriostatic with respect to Gram-positive bacteria.

Advantageously, the bactericidal or bacteriostatic composition or the bactericidal or bacteriostatic agent is incorporated into a food, cosmetic, pharmaceutical, phytosanitary, veterinary or surface treatment composition, such as, for example, a cosmetic and/or dermatological composition for cleansing and/or caring for the skin, in particular in the form of a cream, a gel, a powder, a lotion, a butter especially, a shower gel, soap, shampoo, bath and shower gel, deodorant, antiperspirant, wet wipe, sunscreen formulation or decorative cosmetic formulation.

The invention in some embodiments also relates to a composition, characterized in that it comprises an alkyl acetal or alkyl ether of deoxyhexose or of a deoxyhexose derivative, in which the alkyl group comprises between 11 and 18 carbon atoms, a pharmaceutically acceptable salt, an isomer or a mixture of isomers thereof, said deoxyhexose derivative being a glycosylated and/or hydrogenated and/or dehydrated deoxyhexose, for use thereof as bactericidal or bacteriostatic agent; preferentially, said alkyl ether or alkyl acetal radical is in the 2-O—, 3-O— or 4-O— position, the isomers being preferentially selected from regioisomers and/or diastereoisomers.

The invention in some embodiments also relates to a composition, characterized in that it comprises an alkyl acetal or alkyl ether of deoxyhexose or of a deoxyhexose derivative, in which the alkyl group comprises between 11 and 18 carbon atoms, a pharmaceutically acceptable salt, an isomer or a mixture of isomers thereof, said deoxyhexose derivative being a glycosylated and/or hydrogenated and/or dehydrated deoxyhexose, for use thereof as hygiene or dermatological product for external use.

Typically, a “hygiene product” refers to any product used for cleaning, disinfection or hygiene, including for example a lotion, mousse, spray and liquid but also wipes or any other support capable of being impregnated with the composition according to the invention. The expression “dermatological product” refers to any product intended for application to the skin or the mucous membranes.

### Use in Treating or Preventing a Gram-Positive Bacterial Infection

The invention in some embodiments o also relates to a composition according to an embodiment of the invention, for use thereof in treating or preventing bacterial infections by Gram-positive bacteria.

“Treating” is intended to mean curative treatment (aiming at least to reduce, eradicate, or stop the development of the infection) in a patient. “Preventing” is intended to mean prophylactic treatment (aiming to reduce the risk of the infection appearing) in a patient.

The “patient” may for example be a human or a non-human mammal (for example a rodent (mouse, rat), a feline, a dog or a primate) affected by, or capable of being affected by, bacterial infections and especially Gram-positive bacterial infections. The subject is preferably a human.

The expression “Gram-positive” refers to bacteria which become stained dark blue or violet by Gram staining, as opposed to Gram-negative bacteria which cannot retain the violet stain. The staining technique is well known to those skilled in the art and is based on the cell membrane and cell wall characteristics of the bacterium.

Typically, the Gram-positive bacteria are bacteria from the phylum Firmicutes, typically of the class Bacilli, especially chosen from bacteria of the order Lactobacillales or Bacillales.

According to one embodiment of the invention, the bacteria of the order Bacillales are chosen from the family Alicyclobacillaceae, Bacillaceae, Caryophanaceae, Listeriaceae, Paenibacillaceae, Pasteuriaceae, Planococcaceae, Sporolactobacillaceae, Staphylococcaceae, Thermoactinomycetacea or Turicibacteraceae.

Typically, the bacteria of the family Listeriaceae are for example of the genus Brochothrix or Listeria and may typically be chosen from L. fleischmannii, L. grayi, L. innocua, L. ivanovii, L. marthii, L. monocytogenes, L. rocourtiae, L. seeligeri, L. weihenstephanensis and L. welshimeri.

When the Gram-positive bacteria are bacteria of the family Staphylococcaceae, they are especially chosen from bacteria of the genus Staphylococcus, Gemella, Jeotgalicoccus, Macrococcus, Salinicoccus and Nosocomiicoccus.

The bacteria of the genus Staphylococcus are for example chosen from S. arlettae, S. agnetis, S. aureus, S. auricularis, S. capitis, S. caprae, S. carnosus, S. caseolyticus, S. chromogenes, S. cohnii, S. condimenti, S. delphini, S. devriesei, S. epidermidis, S. equorum, S. fells, S. fleurettii, S. gallinarum, S. haemolyticus, S. hominis, S. hyicus, S. intermedius, S. kloosii, S. leei, S. lentus, S. lugdunensis, S. lutrae, S. massiliensis, S. micron, S. muscae, S. nepalensis, S. pasteuri, S. pettenkoferi, S. piscifermentans, S. pseudintermedius, S. pseudolugdunensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri and S. xylosus.

According to another embodiment of the invention, the bacteria of the order Lactobacillales are chosen from the families Aerococcaceae, Carnobacteriaceae, Enterococcaceae, Lactobacillaceae, Leuconostocaceae and Streptococcaceae.

Typically, the bacteria of the family Enterococcaceae are chosen from bacteria of the genus Bavariicoccus, Catellicoccus, Enterococcus, Melissococcus, Pilibacter, Tetragenococcus or Vagococcus.

The bacteria of the genus Enterococcus are for example chosen from E. malodoratus, E. avium, E. durans, E. faecalis, E. faecium, E. gallinarum, E. hirae, E. solitarius, preferentially E. avium, E. durans, E. faecalis and E. faecium.

The bacteria of the genus Staphylococcus and more particularly S. aureus are responsible for numerous infections of the skin or of the mucous membranes, such as the vaginal or nasal mucous membrane. For example, infections such as folliculitis, abscesses, paronychia, boils, impetigo, infections between the digits, anthrax (staphylococcal anthrax), cellulitis, secondary wound infections, otitis, sinusitis, hidradenitis, infectious mastitis, post-traumatic skin infections or infections of burnt skin.

The bacteria of the genus Enterococcus, and especially E. faecalis, are responsible especially for endocarditis, and infections of the bladder, prostate or epididymis.

The invention in some embodiments also relates to a method for treating or preventing a bacterial infection by Gram-positive bacteria, preferentially an infection of the skin or mucous membranes, by administration, preferentially topical, to an individual who needs it, of a therapeutically effective amount of the composition according to an embodiment of the invention.

In a person infected with a Gram-positive bacterium, “therapeutically effective amount” is intended to mean an amount that is sufficient to prevent the infection worsening or even sufficient to cause the infection to regress. In an uninfected person, the “therapeutically effective amount” is the amount that is sufficient to protect a person who may have come into contact with a Gram-positive bacterium and to avoid the onset of infection caused by this Gram-positive bacterium.

Typically, topical administration is carried out by application, to the skin or to the mucous membranes, of the composition according to an embodiment of the invention.

### Method for Disinfecting or for Preventing Bacterial Colonization of a Substrate

The invention also relates to a method for disinfecting or for preventing bacterial colonization by Gram-positive bacteria of a substrate, comprising bringing the substrate into contact with a composition according to the invention.

Typically, the substrate is any support capable of being colonized by Gram-positive bacteria and capable of transmitting the infection to an animal by contact or by ingestion.

For example, the substrate may be a food of plant or animal origin or a dietary composition comprising such foods, or an extract of these foods, and especially cereals, fruit, vegetables, meat, fish or offal.

The substrate may also be one or more elements chosen from metals, plastics, glass, concrete or stone.

Preferentially, the substrate is a utensil, a tool or an apparatus used in the food industry (cooking utensils, containers, cold-storage system, refrigerator, cold rooms, etc.), in the hospital sector, such as for example surgical instruments or prostheses, or in public transport (public transport handrails, seats, etc.).

The invention in some embodiments also relates to a composition for disinfecting, treating, sterilizing or purifying surfaces. Although they have distinct meanings, the terms “comprising”, “containing”, “including” and “consisting of” have been used interchangeably in the description of the invention, and may be replaced by one another.

The invention will be better understood on reading the following figures and examples, given solely by way of example.

### EXAMPLES

The methyl glycopyranoside acetals were prepared by acetalization or trans-acetalization of the sugars, according to the procedure described previously in patent Ser. No. 13/01375, “Process for preparing long-chain cyclic alkyl acetals based on sugars”. The methyl glycopyranoside alkyl acetals are then reduced using reducing conditions, without acid catalyst, described previously in patent Ser. No. 14/01346. By way of indication, the synthesis of methyl glycopyranoside ethers and acetals is described in detail below.

### EXAMPLE 1: General Procedure for Preparing Methyl Glycopyranoside Alkyl Acetals (A)

The methyl glycopyranoside (2 equivalents) is dissolved in dry THF (10 ml) in the presence of sodium sulfate (1.5 equivalents) in a 100 ml round-bottomed flask under an argon atmosphere. The aldehyde (1 equivalent) is added dropwise over a duration of 1 minute, followed by Amberlyst 15 (20% by weight relative to the aldehyde). The reaction mixture is stirred with a magnetic stirrer at reflux (65° C.) for 3 hours. After returning to room temperature, the reaction mixture is filtered, washed with ethyl acetate (2×25 ml) and the filtrate is concentrated under reduced pressure. The residue is purified by chromatography on a silica gel column (AcOEt/cyclohexane) to give methyl glycopyranoside alkyl acetals.

### Example 1a

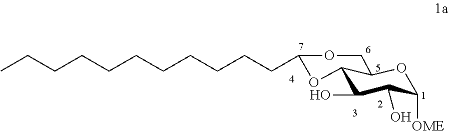

Methyl 4,6-O-dodecylidene-α-D-glucopyranoside (1a): The compound 1a was prepared from methyl α-D-glucopyranoside (3.22 g, 16.6 mmol) and dodecanal (1.52 g, 8.3 mmol) according to procedure (A). After reaction, the residue was purified by chromatography on a silica gel column (EtOAc/cyclohexane 60:40) to give 1a (0.77 g, 26%) in the form of a white solid. Melting point=69° C.; ¹NMR (300 MHz, CDCl₃) δ_{H}: 0.86; (3H, t, J=7, CH₃), 1.17-1.32; (16H, m, 8CH₂), 1.33-1.47; (2H, m, CH₂), 1.53-1.74; (2H, m, CH₂), 2.64; (2H, br s, OH³+OH²), 3.24; (1H, t, J=9.0, CH³), 3.41; (3H, s, OCH₃), 3.49-3.68; (3H, m, CH⁵+CH⁶+CH²), 3.84; (1H, t, J=9.0, CH⁴), 4.10; (1H, dd, J=10.0 and 5.0, CH⁶), 4.52; (1H, t, J=5.0, CH⁷), 4.74; (1H, d, J=4.0, CH); ¹³C NMR (75 MHz, CDCl₃) δ_{C}: 14.24; (CH₃), 22.80; (CH₂), 24.20; (CH₂), 29.46; (CH₂), 29.58; (CH₂), 29.62; (CH₂), 29.67; (CH₂), 29.74; (CH₂), 29.76; (CH₂), 32.03; (CH₂), 34.36; (CH₂), 55.57; (OCH₃), 62.63; (CH⁵), 68.57; (CH₂⁶), 71.81; (CH⁴), 73.02; (CH²), 80.46; (CH³), 99.85; (CH¹), 102.84; (CH⁷); IR vₘₐₓ: 3388 (OH), 2921, 2852, 1466, 1378, 1089, 1063, 1037, 991; HRMS (ESI⁺) calculated for C₁₉H₃₆NaO₆: 383.2404 [M+Na]⁺; measured: 383.2398 (+1.6 ppm); Rf=0.30 (EtOAc/cyclohexane 60:40).

### Example 1b

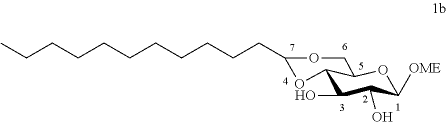

Methyl 4,6-O-dodecylidene-β-D-glucopyranoside (1b): The compound 1b was prepared from methyl β-D-glucopyranoside (5.00 g, 25.7 mmol) and dodecanal (2.37 g, 12.8 mmol) according to procedure (A). After reaction, the residue was purified by chromatography on a silica gel column (EtOAc/cyclohexane, from 30:70 to 50:50) to give 1b (1.30 g, 28%) in the form of a white solid. Melting point=84° C.; ¹H NMR (300 MHz, CDCl₃) δ_{H}: 0.87 ;(3H, t, J=6.7, CH₃), 1.25; (16H, app br s, 8 CH₂), 1.34-1.45; (2H, m, CH₂), 1.53-1.73; (2H, m, CH₂), 3.25-3.34; (2H, m, CH²+CH⁵), 3.44; (1H, dd, J=9.0, 7.0, CH³), 3.56; (4H, s, CH₂⁶+OCH₃), 3.73; (1H, m, CH⁴), 4.18; (1H, dd, J=10.4, 4.4, CH₂⁶), 4.28; (1H, d, J=7.7, CH), 4.54; (1H, t, J=5.1, CH⁷); ¹³C NMR (75 MHz, CDCl₃) δ_{C}: 14.13; (CH₃), 22.70; (CH₂), 24.14; (CH₂), 29.35; (CH₂), 29.45; (CH₂), 29.50; (CH₂), 29.56; (CH₂), 29.63; (CH₂), 29.65; (CH₂), 31.92; (CH₂), 34.23; (CH₂), 55.51; (OCH₃), 66.21; (CH⁵), 68.21; (CH₂⁶), 73.19; (CH⁴), 74.61; (CH²), 80.00; (CH³), 102.83; (CH⁷), 104.07; (CH¹); IR vₘₐₓ: 3650 (OH), 2950, 2824, 2867, 2159, 2028, 1112; HRMS (ESI⁺) calculated for C₁₉H₃₆NaO₆: 383.2404 [M+Na]⁺; measured: 383.2395 (+2.3 ppm). Rf=0.30 (EtOAc/cyclohexane 40:60)

### Example 1c

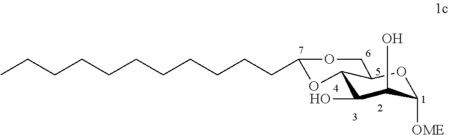

Methyl 4,6-O-dodecylidene-α-D-mannopyranoside (1c): The compound 1c was prepared from methyl α-D-mannopyranoside (4.00 g, 20.5 mmol) and dodecanal (3.45 g, 18.7 mmol) according to procedure (A). After reaction, the reaction medium is concentrated under reduced pressure and dissolved in CH₂Cl₂. The organic phase is washed with water (3×100 ml), with a saturated NaCl solution (2×100 ml), dried (Na₂SO₄) and concentrated under reduced pressure. The residue was purified by chromatography on a silica gel column (EtOAc/cyclohexane, from 20:80 to 50:50) to give 1c (0.73 g, 11%) in the form of a white solid. Melting point=104° C.; ¹H NMR (300 MHz, CDCl₃) δ_{H}: 0.88 (3H, t, J=6.9, CH₃), 1.17-1.32; (16H, m, 8 CH₂), 1.37-1.42; (2H, m, CH₂), 1.58-1.68; (2H, m, CH₂), 3.37; (3H, s, OCH₃), 3.53-3.72; (3H, m, CH³+CH⁵+CH⁶), 3.98; (1H, dd, J=9.0, 3.7, CH²), 4.13; (1H, dd, J=3.6, 1.4, CH⁴), 4.58; (1H, dd, J=8.8, 2.9, CH⁶), 4.10; (1H, t, J=5.1, CH⁷), 4.73; (1H, d, J=1.3, CH¹); ¹³C NMR (75 MHz, CDCl₃) δ_{C}: 14.13; (CH₃), 22.69; (CH₂), 24.10; (CH₂), 29.35; (CH₂), 29.46; (CH₂), 29.51; (CH₂), 29.56; (CH₂), 29.63; (CH₂), 29.65; (CH₂), 31.92; (CH₂), 34.40; (CH₂), 55.05; (OCH₃), 63.00; (CH⁵), 68.38; (CH₂⁶), 68.81; (CH²), 70.82; (CH⁴), 78.23; (CH³), 101.15; (CH¹), 103.06; (CH⁷); IR vₘₐₓ: 3380 (OH), 2924, 2852, 1466, 1156, 1029, 682; HRMS (ESI⁺) calculated for C₁₉H₃₆NaO₆: 383.2404 [M+Na]⁺; measured: 383.2396 (+2.2 ppm). Rf=0.2 (cyclohexane/EtOAc, 70:30).

### Example 1d

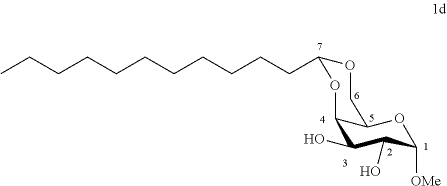

Methyl 4,6-O-dodecylidene-α-D-galactopyranoside (1d): The compound 1d was prepared from methyl a-D-galactopyranoside (5.00 g, 25.7 mmol) and dodecanal (2.37 g, 12.9 mmol) according to procedure (A). After reaction, the reaction medium is concentrated under reduced pressure to give 1d (2.30 g, 45%) in the form of a white solid without purification by chromatography. Melting point=115° C.; ¹H NMR (300 MHz, CDCl₃) δ_{H}: 0.89; (3H, t, J=6.7, CH₃), 1.15-1.50; (18H, m, 9 CH₂), 1.61-1.71; (2H, m, CH₂), 3.45; (3H, s, OCH₃), 3.61; (1H, app. s, CH⁵), 3.77-3.94; (3H, m, CH⁴+CH²CH⁶), 4.04; (1H, d, J=2.5, H³), 4.14; (1H, dd, J=12.5, 1.4, CH⁶), 4.59; (1H, t, J=5.2, CH⁷), 4.91; (1H, d, J=3.2, CH); ¹³C NMR (75 MHz, CDCl₃) δ_{C}: 14.06; (CH₃), 22.50; (CH₂), 23.49; (CH₂), 29.27; (CH₂), 29.34; (CH₂), 29.41; (CH₂), 29.48; (CH₂), 29.55; (CH₂), 29.61; (CH₂), 31.97; (CH₂), 34.47; (CH₂), 55.66; (OCH₃), 62.45; (CH⁵), 68.92; (CH₂⁶), 69.82; (CH²), 69.92; (CH⁴), 75.42; (CH³), 100.1; (CH⁷), 102.1; (CH); IR vₘₐₓ: 3414, 3328 (OH), 2916, 2850, 2160, 1121, 1032; HRMS (ESI⁺) calculated for C₁₉H₃₆NaO₆ 383.2404 [M+Na]⁺; measured: 383.2389 (+4.0 ppm). Rf=0.6 (EtOAc/cyclohexane 60:40).

### Example 1e

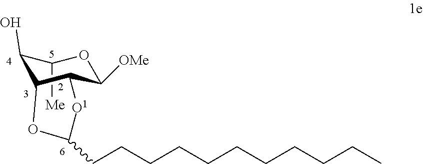

Methyl 2,3-O-dodecylidene-α-L-rhamnopyranoside (le): The compound le was prepared from methyl α-L-rhamnopyranoside (1.00 g, 5.60 mmol) and dodecanal (0.94 g, 5.10 mmol) according to procedure (A). After reaction, the reaction medium is concentrated under reduced pressure and dissolved in CH₂Cl₂. The organic phase is washed with water (3×100 ml), with a saturated NaCl solution (2×100 ml), dried (Na₂SO₄) and concentrated under reduced pressure. The residue was purified by chromatography on a silica gel column (EtOAc/cyclohexane, from 0:100 then from 10:90 to 100:0). An inseparable 53:47 mixture of two diastereoisomers of 1e (0.85 g, 49%) was obtained in the form of a beige solid. Melting point=46° C.; ¹H NMR (300 MHz, CDCl₃) δ_{H} for the mixture of diastereoisomers: 0.88; (6H, t, J=6.7, 2×CH₃), 1.25-1.32; (42H, m, 18(CH₂)+2×CH₃), 1.57-1.63; (2H, m, CH₂), 1.67-1.74; (2H, m, CH₂), 3.34-3.42; (2H, m, 2×CH⁴), 3.38; (3H, s, OCH₃), 3.39; (3H, s, OCH₃), 3.65-3.67; (2H, m, 2×CH⁵), 3.93-4.00; (2H, m, CH²+CH³), 4.00-4.08; (1H, m, CH²), 4.18; (1H, dd, J=7.4, 5.4, CH³), 4.85; (1H, s, CH¹), 4.88; (1H, s, CH¹), 4.98; (1H, t, J=5.0, CH⁶), 5.24; (1H, t, J=4.8, CH⁶); ¹³C NMR (75 MHz, CDCl₃) δ_{C} for the mixture of diastereoisomers: 14.13; (2×CH₃), 17.41; (CH₃), 17.56; (CH₃), 22.70; (2 CH₂), 23.77; (CH₂), 24.23; (CH₂), 29.35; (2 CH₂), 29.49; (CH₂), 29.50; (CH₂), 29.53; (2 CH₂), 29.56; (2 CH₂), 29.62; (2 CH₂), 29.65; (2 CH₂), 31.92; (2 CH₂), 34.90; (CH₂), 35.41; (CH₂), 54.96; (2 OCH₃), 65.24; (CH⁵), 65.87; (CH⁵), 71.57; (CH⁴), 74.94; (CH⁴), 75.17; (CH³), 77.37; (CH²), 77.40; (CH²), 78.99; (CH³), 97.96; (CH¹), 98.28; (CH¹), 104.2; (CH⁶), 104.3 (CH⁶); IR vₘₐₓ: 3650, 3238; (OH), 2921, 2852, 2159, 2029, 1136, 1029; HRMS (ESI⁺) calculated for C₁₉H₃₆NaO₅ 367.2455 [M+Na]⁺; measured: 367.2452 (+0.9 ppm). Rf=0.5 (EtOAc/cyclohexane 50:50).

### EXAMPLE 2: General Procedure for Preparing a Mixture of Regioisomers of Methyl Glycopyranoside Alkyl Ethers (B)

The methyl glycopyranoside alkyl acetal (3 mmol) is dissolved in cyclopentyl methyl ether (CPME, 30 ml) in a 100 ml stainless steel autoclave and 5%-Pd/C (0.45 g, 5 mol % of palladium) is then added. The reactor is hermetically closed, purged three times with hydrogen and hydrogen is introduced at a pressure of 30 bar. The reaction mixture is stirred mechanically and is heated at 120° C. for 15 hours. After cooling to room temperature, the hydrogen pressure is released and the reaction mixture is diluted in absolute ethanol (100 ml) and filtered (0.01 μm Millipore Durapore filter). The filtrate is concentrated under reduced pressure to give the mixture of regioisomers of methyl glycopyranoside alkyl ethers.

### Example 2a

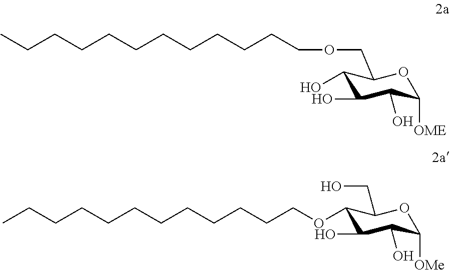

Methyl 6-O-dodecyl-α-D-glucopyranoside (2a) and methyl 4-O-dodecyl-α-D-glucopyranoside (2a′): The compounds 2a and 2a′ were prepared from methyl 4,6-O-dodecylidene-α-D-glucopyranoside 1a (5.00 g, 14 mmol) according to the general procedure (B). A 73:27 mixture of 2a and 2a′ (2.52 g, 51%) was obtained in the form of a white solid. In order to facilitate characterization of the compounds, the regioisomers of the mixture may be separated by chromatography on a silica gel column (EtOAc/cyclohexane, from 50:50 to 100:0 then EtOH/EtOAc 10:90). 2a: White solid. ¹H NMR (300 MHz, CDCl₃) δ_{H}: 0.87; (3H, t, J=7, CH₃ alkyl), 1.09-1.44; (18H, m, 9(CH₂) alkyl), 1.47-1.70; (2H, m, CH₂) alkyl, 3.41; (3H, s, OCH₃), 3.43-3.84; (7H, m), 4.21; (3H, br s, OH), 4.74; (1H, d, J=4, anomeric CH); ¹³C NMR (75 MHz, CDCl₃) δ_{C}: 14.25; (CH₃), 22.82; (CH₂), 26.17; (CH₂), 29.50; (CH₂), 29.67; (CH₂), 29.73; (CH₂), 29.77; (CH₂), 29.80; (2CH₂), 29.83; (CH₂), 32.06; (CH₂), 55.35; (OCH₃), 70.33; (CH), 70.51; (CH₂), 71.23; (CH), 72.10; (CH), 72.30; (CH₂), 74.49; (CH), 99.57; (CH); IR vₘₐₓ: 3402 (OH), 2918, 2851, 1467, 1370, 1057, 1015, 902; HRMS (ESI⁺) calculated for C₁₉H₃₈NaO₆: 385.2561 [M+Na]⁺; measured: 385.2558; (+0.6 ppm); Rf=0.16 (EtOAc/EtOH 10:1). 2a′: white solid. ¹H NMR (300 MHz, CDCl₃) δ_{H}: 0.87; (3H, t, J=7, CH₃ alkyl), 1.14-1.42; (18H, m, 9(CH₂) alkyl), 1.47-1.71; (2H, m, CH₂ alkyl), 2.16; (3H, br s, OH), 3.24; (1H, t, J =10); 3.41; (3H, s, OCH₃), 3.49; (1H, dd, J==10 and 4), 3.54-3.66; (2H, m), 3.69-3.91; (4H, m), 4.74; (1H, d, J=4, anomeric CH); ¹³C NMR (75 MHz, CDCl₃) δ_{C}: 14.26; (CH₃), 22.83; (CH₂), 26.20; (CH₂), 29.49; (CH₂), 29.64; (CH₂), 29.74; (2CH₂), 29.77; (CH₂), 29.80; (CH₂), 30.47; (CH₂), 32.06; (CH₂), 55.46; (OCH₃), 62.15; (CH₂), 70.99; (CH), 72.81; (CH), 73.28; (CH₂), 75.05; (CH), 77.94; (CH), 99.20; (CH); IR vₘₐₓ: 3295; (OH), 2913, 2848, 1739, 1469, 1370, 1114, 1067, 1042, 993; HRMS (ESI⁺) calculated for C₁₉H₃₈NaO₆: 385.2561 [M+Na]⁺; measured: 385.2574 (−3.5 ppm); Rf=0.24 (EtOAc/EtOH 10:1).

### Example 2b

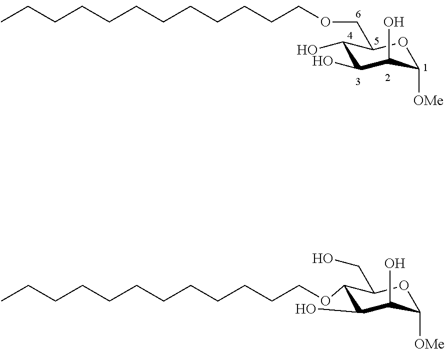

Methyl 6-O-dodecyl-α-D-mannopyranoside (2b) and methyl 4-O-dodecyl-α-D-mannopyranoside (2b′): The compounds 2b and 2b′ were prepared from methyl 4,6-O-dodecylidene-α-D-mannopyranoside 1c (0.70 g, 1.94 mmol) according to the general procedure (B). After reaction, the residue was purified by chromatography on a silica gel column (EtOAc/cyclohexane, 40:60). An inseparable 75:25 mixture of 2b and 2b′ (0.24 g, 34%) was obtained in the form of a colorless oil. ¹H NMR (300 MHz, CDCl₃) δ_{H} for the predominant regioisomer 2b: 0.88; (3H, t, J=6.7, CH₃), 1.20-1.35; (18H, m, 9 CH₂), 1.55-1.61; (2H, m, CH₂), 3.35; (3H, s, OCH₃), 3.44-3.57; (2H, m, OCH₂), 3.60-3.98; (6H, m, CH²+CH³+CH⁴+CH⁵+CH₂⁶), 4.73; (1H, d, J=1.5, CH); ¹³C NMR (75 MHz, CDCl₃) δ_{C} for the predominant regioisomer 2b: 14.06; (CH₃), 22.63; (CH₂), 25.95; (CH₂), 29.30; (CH₂), 29.42; (CH₂), 29.44; (CH₂), 29.54; (CH₂), 29.57; (CH₂), 29.58; (CH₂), 29.61; (CH₂), 31.86; (CH₂), 54.96; (OCH₃), 69.50; (CH⁵), 69.65; (CH⁴), 70.37; (CH²), 71.12; (CH₂⁶), 71.67; (CH₃), 72.14; (OCH₂), 100.7; (CH); IR vₘₐₓ: 3650, 3238 (OH), 2921, 2852, 2159, 2029, 1976, 1156; HRMS (ESI⁺) calculated for C₁₉H₃₈NaO₆: 385.2561 [M+Na]⁺; measured: 385.2555 (+1.5 ppm); Rf=0.22 (cyclohexane/EtOAc 60:40).

### Example 2c

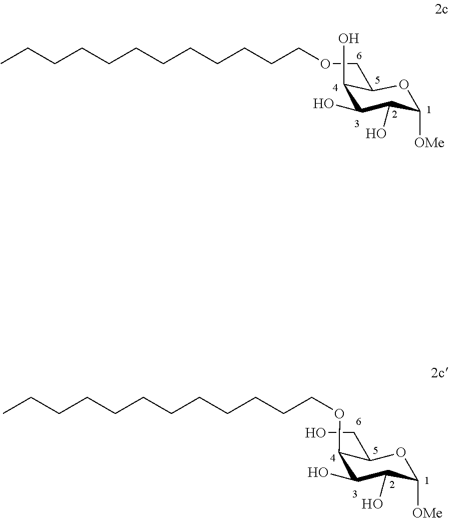

Methyl 6-O-dodecyl-α-D-galactopyranoside (2c) and methyl 4-O-dodecyl-α-D-galactopyranoside (2c′): The compounds 2c and 2c′ were prepared from methyl 4,6-O-dodecylidene-α-D-galactopyranoside 1d (0.69 g, 1.90 mmol) according to the general procedure (B). After reaction, the residue was purified by chromatography on a silica gel column (EtOAc/cyclohexane, 50:50). An inseparable 90:10 mixture of 2c and 2c′ (0.19 g, 27%) was obtained in the form of a white solid. Melting point=110° C.; ¹H NMR (300 MHz, CDCl₃) δ_{H} for the predominant regioisomer 2c: 0.87; (3H, t, J=6.6, CH₃), 1.24; (18H, br s, 9 CH₂), 1.55-1.60; (2H, m, CH₂), 3.41; (3H, s, OCH₃), 3.48; (2H, t, J=6.7, OCH₂), 3.67-3.90; (5H, m, 3 CH+CH₂), 4.04-4.05; (1H, m, CH), 4.83; (1H, d, J=3.5, CH); ¹³C NMR (75 MHz, CDCl₃) δ_{C} for the predominant regioisomer 2c′: 14.24; (CH₃), 22.81; (CH₂), 26.17; (CH₂), 29.47; (CH₂), 29.59; (CH₂), 29.61; (CH₂), 29.70; (CH₂), 29.74; (CH₂), 29.76; (2 CH₂), 29.78; (CH₂), 32.44; (CH₂), 55.59; (OCH₃), 69.68; (CH), 70.47; (CH), 71.11; (CH), 71.34; (CH), 72.30; (CH₂), 99.84; (CH); IR vₘₐₓ: 3651, 3250; (OH), 2917, 2849, 2493, 2430, 2159, 2029, 1976, 1042; HRMS (ESI⁺) calculated for C₁₉H₃₈NaO₆: 385.2561 [M+Na]⁺; measured: 385.2548 (+3.2 ppm); Rf=0.30 (cyclohexane/EtOAc 40:60).

### Example 2d

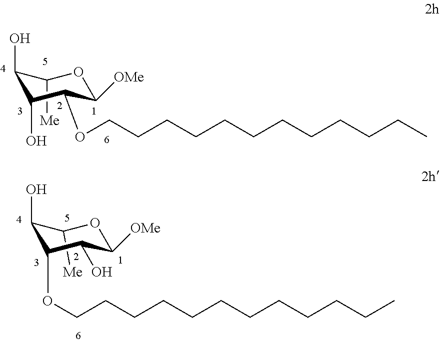

Methyl 2-O-dodecyl-α-L-rhamnopyranoside (2d) and methyl 3-O-dodecyl-α-L-rhamnopyranoside (2d′): The compounds 2d and 2d′ were prepared from methyl 2,3-O-dodecylidene-α-L-rhamnopyranoside 1e (0.70 g, 2.03 mmol) according to the general procedure (B). After reaction, the residue was purified by chromatography on a silica gel column (EtOAc/cyclohexane, 40:60). An inseparable 93:7 mixture of 2d and 2d′ (0.19 g, 27%) was obtained in the form of a colorless oil. ¹H NMR (300 MHz, CDCl₃) 6_{H} for the predominant regioisomer 2d: 0.87; (3H, t, J=6.7, CH₃), 1.18-1.35; (21H, m, 9 (CH₂)+CH₃), 1.53-1.59; (2H, m, CH₂), 2.35; (2H, br s, OH), 3.31-3.47; (5H, m, CH³+CH⁶+OCH₃), 3.52; (1H, dd, J=3.9, 1.3, CH²), 3.54-3.62; (1H, m, CH⁵), 3.62-3.71; (2H, m, CH⁶+CH⁴), 4.71; (1H, app. s, CH); ¹³C NMR (75 MHz, CDCl₃) δ_{C} for the predominant regioisomer 2d′: 14.11 (CH₃), 17.54; (CH₃), 22.68; (CH₂), 25.99; (CH₂), 29.34; (CH₂), 29.41; (CH₂), 29.56; (CH₂), 29.59; (CH₂), 29.62; (CH₂), 29.65; (CH₂), 29.80; (CH₂), 31.91; (CH₂), 54.75; (OCH₃), 67.38; (CH⁵), 71.24; (CH₂), 71.51; (CH⁴), 74.07; (CH³), 78.53; (CH²), 97.83; (CH); IR vₘₐₓ: 3650, 3238; (OH), 2921, 2852, 2519, 2029, 2029, 1976, 1070; HRMS (ESI⁺) calculated for C₁₉H₃₈NaO₅: 369.2611 [M+Na]⁺; measured: 369.2605 (+1.8 ppm); Rf=0.51 (cyclohexane/EtOAc 60:40).

### EXAMPLE 3: Measurement of the Bacteriostatic Properties of Acetal and Ether Derivatives of C12 Monosaccharides on Gram-Positive Bacteria

Since the best results were observed with compounds having a C12 alkyl group, assays were carried out on a broader panel of Gram-positive strains with compounds obtained according to examples 1 and 2.

### 3.1 Materials and Methods

### 3.1.1 The Compounds of Interest Tested

Methyl Glucopyranoside Acetals
Methyl 4,6-O-dodecylidene-α-D-glucopyranoside (1a)
Methyl 4,6-O-dodecylidene-β-D-glucopyranoside (1b)

Mixture of Methyl Glycopyranoside Ethers
Methyl 6-O-dodecyl-α-D-glucopyranoside (2a) and methyl 4-O-dodecyl-α-D-glucopyranoside (2a′)
Methyl 6-O-dodecyl-α-D-mannopyranoside (2b) and methyl 4-O-dodecyl-α-D-mannopyranoside (2b′)
Methyl 6-O-dodecyl-α-D-galactopyranoside (2c) and methyl 4-O-dodecyl-α-D-galactopyranoside (2c′)
Methyl 2-O-dodecyl-α-L-rhamnopyranoside (2d) and methyl 3-O-dodecyl-α-L-rhamnopyranoside (2d′)

Mixture of Sorbitan Ethers
3-O-Dodecyl-1,4-D-sorbitan, 5-O-dodecyl-1,4-D-sorbitan and 6-O-dodecyl-1,4-D-sorbitan

3.1.2 The Gram-Positive Bacteria Studied

The strains tested are reference strains and cultures that are multi-antibiotic resistant; these are clinical strains that were isolated at the “Hospice de Lyon” and are as follows:
Staphylococcus S. aureus: ATCC® 29213™, ATCC 25923,
Methicillin-resistant S. aureus staphylococcus strains (Lac-Deleo USA 300), (MU 3), (HT 2004-0012), LY 199-0053, (HT 2002-0417), (HT 2006-1004),
Daptomycin-resistant S. aureus staphylococcus strains (ST 2015-0188) (ST 2014 1288), (ST 2015-0989).
Enterococci: E. faecalis (ATCC® 29212™, clinical strains of E. faecalis enterococcus, isolated from urine: strain 015206179901 (hereinafter 9901), strain 015205261801 (hereinafter 1801)
Enterococci: E. faecium (CIP 103510), clinical strains of E. faecium enterococcus: Van A 0151850763 (hereinafter Van A); strain 015 205731401 (hereinafter 1401),
Listeria: L. monocytogenes (CIP 103575), clinical strains isolated from blood culture (015189074801, LM1), strain isolated from cerebrospinal fluid (015170199001, LM2), clinical strain isolated from blood culture (015181840701, LM3).

### 3.1.3 Preparation of the Inoculum

The cultures studied, freshly isolated (after incubation on blood agar at 37° C. for 18 h), are taken up in sterile water (10 ml) until a 0.5 McFarland (Mc), i.e. 1 to 2×10⁸ CFU (bacteria)/cm³, suspension is obtained. The bacterial suspension was then diluted in order to obtain a final concentration of 1×10⁶ CFU/cm³.

### 3.1.4 Preparation of Multi-Well Plates for Observing the MIC

Each well contains a final identical amount of Mueller-Hinton medium (rich medium enabling bacterial culture) and of bacteria of 0.5×10⁶ CFU/cm³.

The compounds of interest to be tested are dissolved in ethanol or DMSO at 25 mg/ml before being diluted, with the dilution being doubled each time, to different concentrations. On the multi-well plate, a first series was provided comprising the culture medium without the compound of interest to be tested. This corresponds to the growth control (control wells). These controls serve as a reference for comparing bacterial growth with those of the following wells comprising different concentrations of the compound of interest to be tested. The second series of wells comprises the parent solution of the compound of interest to be tested for a concentration in the well of 256 mg/l (7 mM). Each series of wells was diluted, with the dilution being doubled each time, until the last series for a final concentration of 0.25 mg/l (0.0007 mM). Each concentration is duplicated within the same plate. The plate is incubated for 18 h at 37° C. Observation after incubation shows cloudiness in the control wells (a sign of bacterial growth). In the case of antibacterial activity, bacterial growth is inhibited which is reflected by the absence of appearance of cloudiness or bacterial pellet.

The minimum inhibitory growths (MIC) are determined on Gram-positive bacterial strains according to the recommendations of the “Clinical Laboratory Standards Institute” (Clinical-Laboratory-Standards-Institute, 6th ed. Approved standard M100-S17. CLSI, Wayne, Pa., 2007).

### 3.1.5 Preparation of the Inoculum

The cultures studied, freshly isolated (after incubation on blood agar at 37° C. for 18 h), are taken up in sterile water (10 ml) until a 0.5 McFarland (Mc), i.e. to 10⁸ CFU (bacteria)/cm³, suspension is obtained. The bacterial suspension was then diluted in order to obtain a final concentration of 10⁶ CFU/cm³.

### 3.2 Results

### 3.2.1 Results for the Strains of the Genus Staphylococcus

According to the observation of the 96-well microplates, all the acetal or ether monosaccharide derivatives are active against the tested Staphylococcus strains (8<MIC<64 mg/l) with the exception of the galactose ether (C12-Eth-α-MeGalac) and the glucose α-acetal (C12-Ac-α-MeGlu) (MIC>256 mg/l).

During the analysis of the results (table 6), it will be noted that all these derivatives do indeed comprise a C12 carbon-based chain. Yet, only some of them are active against the Staphylococcus strains tested (8<MIC<64 mg/l). In addition, when molecules 1a and 1b are compared, it is observed that these molecules only differ from one another in terms of their anomeric state; and only one of them has effective antibacterial activity.

TABLE 6
Antimicrobial results for the methyl and sorbitan glycopyranoside acetal and ether derivatives on different
S. aureus staphylococcal strains: Minimum inhibitory concentration (MIC) in mg/l.

| | Staphylococcus | | | | | | | | | | |
|---|---|---|---|---|---|---|---|---|---|---|---|
| | | | | | HT | LY | HT | HT | ST | ST | ST |
| | ATCC | ATCC | USA | MU | 2004- | 199- | 2002 | 2006 | 2015 | 2014 | 2015 |
| | 25923 | 29213 | 300 | 3 | 0012 | 0053 | 0417 | 1004 | 0188 | 1288 | 0989 |
| 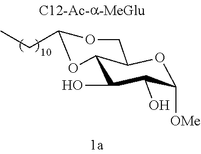 | 256 | 256 | 256 | 256 | 256 | 256 | 256 | 256 | / | / | / |
| 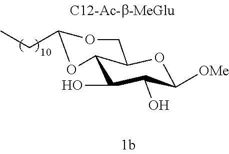 | 64 | 64 | 64 | 64 | | 64 | 128 | 64 | 64 | 64 | 64 |
| 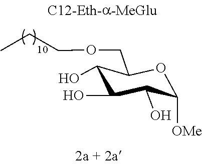 | 16 | 32 | 32 | 32 | 32 | 16 | 16 | 32 | 32 | 32 | 32 |
| 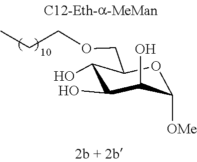 | 32 | 32 | 32 | 64 | 32 | 32 | 32 | 64 | 64 | 32 | 64 |
| 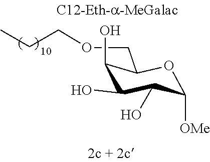 | 124 | 256 | 256 | 256 | 128 | 246 | 256 | 256 | 256 | 256 | 256 |
| 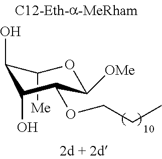 | 32 | 16 | 32 | 64 | 16 | 32 | 32 | 32 | 64/32 | 32 | 64 |
| 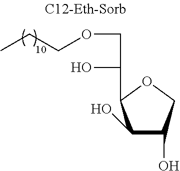 | 32 | 32 | 32 | 64 | 32 | 32 | 32 | 32 | 64 | 64 | 256 |

Similarly, if molecules 1a and 2a+2a′ are compared, which differ by the ether or acetal bond, only one of these molecules has effective antibacterial activity. Finally, the nature of the sugar is also capable of changing the antibacterial activity of the molecule. Thus, it is noted that C12-Eth-α-MeGlu, C12-Eth-α-MeRham and C12-Eth-α-MeMan have antibacterial activity compared to C12-Eth-α-MeGalac. This information clearly indicates that the bacterial activity of a molecule is dependent in a combined manner on the nature of the sugar, the configuration of the anomeric carbon and the nature of the bond to the alkyl chain.

### Results for the Strains of the Genus Enterococcus

TABLE 7
Antimicrobial results for the derivatives of sugar ethers and sugar
acetals and sorbitan acetals on different enterococcal strains.
Minimum inhibitory concentration (MIC) in mg/l.

| | Enterococcus | | | | | |
|---|---|---|---|---|---|---|
| | ATCC | | CIP | | | |
| | 29212 | Van A | 103510 | 1401 | 9901 | 1801 |
| 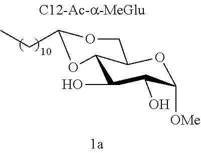 | 256 | 256 | 256 | / | / | / |
| 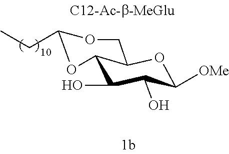 | 64 | 32 | 32 | 16 | 32 | 8 |
| 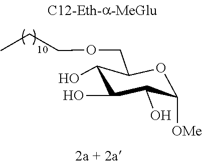 | 16 | 16 | 16 | 8 | 16 | 8 |
| 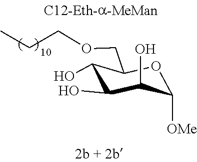 | 16 | 16 | 32 | 16 | 32 | 16 |
| 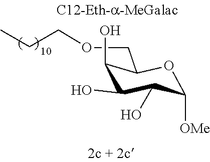 | 64 | 124 | 256 | 32 | 64 | 8 |
| 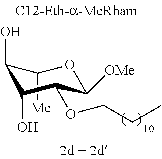 | 16 | 16 | 16 | 8 | 16 | 16 |
| 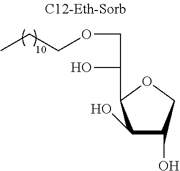 | 8 | 16 | 16 | 8 | 16 | 8 |

Good antibacterial activity is observed for all the Enterococcus strains; 32<MIC<8 mg/l for all the molecules tested with the exception of C12-Ac-α-MeGlu and C12-Eth-α-MeGalac.

### Results for the Strains of the Genus Listeria

TABLE 8
Antimicrobial results for the derivatives of sugar ethers and
sugar acetals and sorbitan acetals on different strains
of Listeria, minimum inhibitory concentration (MIC) in mg/l.

| | Listeria | | | |
|---|---|---|---|---|
| | CIP 103575 | LM1 | LM2 | LM3 |
| 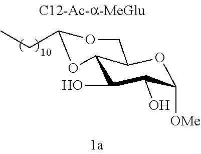 | 64 | / | / | / |
| 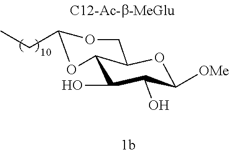 | 16 | 16 | 16 | 64 |
| 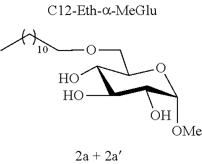 | 8 | 8 | 8 | 8 |
| 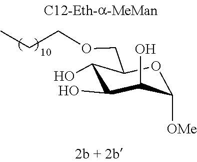 | 32 | 8 | 16 | 16 |
| 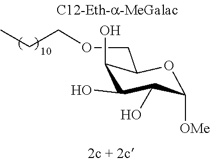 | 64 | 64 | 64 | 64 |
| 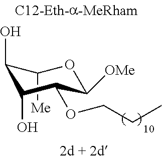 | 32 | 32 | 32 | 32 |
| 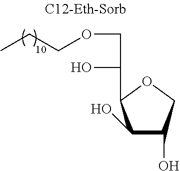 | 32 | 16 | 32 | 32 |

With the exception of the compounds C12-Ac-α-MeGlu and C12-Eth-α-MeGalac, it will be noted that good antibacterial activity is observed on all the strains of Listeria; 64<MIC<8 mg/l for all the molecules tested.

1. A composition, characterized in that it comprises
a deoxyhexose alkyl ether or alkyl acetal in which the alkyl group comprises between 11 and 18 carbon atoms, and
a pharmaceutically acceptable salt, an isomer, or a mixture of isomers of the deoxyhexose alkyl ether or alkyl acetal, the isomers being chosen from regioisomers and/or diastereoisomers.

2. The composition as claimed in claim 1, characterized in that the deoxyhexose is glycosylated and/or hydrogenated and/or dehydrated.

3. The composition as claimed in claim 1, characterized in that the alkyl group comprises 11 to 13 carbon atoms.

4. The composition as claimed in claim 1, characterized in that the deoxyhexose derivative is a rhamnopyranoside.

5. The composition as claimed in claim 1, characterized in that the deoxyhexose alkyl ether or alkyl acetal is a deoxyhexose alkyl monoacetal or monoether.

6. The composition as claimed in claim 1, for use as bactericidal or bacteriostatic agent with respect to Gram-positive bacteria.

7. The composition as claimed in claim 6, characterized in that the Gram-positive bacteria are bacteria from the phylum Firmicutes.

8. The composition as claimed in claim 6, characterized in that the Gram-positive bacteria are bacteria of the order Bacillales chosen from the family Alicyclobacillaceae, Bacillaceae, Caryophanaceae, Listeriaceae, Paenibacillaceae, Pasteuriaceae, Planococcaceae, Sporolactobacillaceae, Staphylococcaceae, Thermoactinomycetacea and Turicibacteraceae, and/or the Gram-positive bacteria are bacteria of the order Lactobacillales chosen from the family Aerococcaceae, Carnobacteriaceae, Enterococcaceae, Lactobacillaceae, Leuconostocaceae and Streptococcaceae.

9. The composition as claimed in claim 8, characterized in that the Gram-positive bacteria are bacteria of the family Listeriaceae, such as a bacterium of the genus Brochothrix or Listeria typically, chosen from L. fleischmannii, L. grayi, L. innocua, L. ivanovii, L. marthii, L. monocytogenes, L. rocourtiae, L. seeligeri, L. weihenstephanensis and L. welshimeri and/or the Gram-positive bacteria are bacteria of the family Staphylococcaceae chosen from bacteria of the genus Staphylococcus, Gemella, Jeotgalicoccus, Macrococcus, Salinicoccus and Nosocomiicoccus.

10. The composition as claimed in claim 9, characterized in that the Gram-positive bacteria are bacteria of the genus Staphylococcus, chosen from S. arlettae, S. agnetis, S. aureus, S. auricularis, S. capitis, S. caprae, S. carnosus, S. caseolyticus, S. chromogenes, S. cohnii, S. condimenti, S. delphini, S. devriesei, S. epidermidis, S. equorum, S. felis, S. fleurettii, S. gallinarum, S. haemolyticus, S. hominis, S. hyicus, S. intermedius, S. kloosii, S. leei, S. lentus, S. lugdunensis, S. lutrae, S. massiliensis, S. microti, S. muscae, S. nepalensis, S. pasteuri, S. pettenkoferi, S. piscifermentans, S. pseudintermedius, S. pseudolugdunensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri and S. xylosus.

11. The composition as claimed in claim 8, characterized in that the Gram-positive bacteria are bacteria of the family Enterococcaceae, chosen from the bacteria of the genus Bavariicoccus, Catellicoccus, Enterococcus, Melissococcus, Pilibacter, Tetragenococcus, or Vagococcus.

12. The composition as claimed in claim 11, characterized in that the Gram-positive bacteria are bacteria of the genus Enterococcus, chosen from E. malodoratus, E. avium, E. durans, E. faecalis, E. faecium, E. gallinarum, E. hirae, E. solitarius, preferentially E. avium, E. durans, E. faecalis and E. faecium.

13. The composition as claimed in claim 1, characterized in that it is incorporated into a food, cosmetic, pharmaceutical, phytosanitary, veterinary or surface treatment composition.

14. The composition as claimed in claim 1, for use thereof as a hygiene or dermatological product for external use and/or for use thereof in treating or preventing bacterial infections by Gram-positive bacteria.

15. The composition as claimed in claim 14, in which the infection by Gram-positive bacteria is an infection of the skin or the mucous membranes, preferentially an infection chosen from folliculitis, an abscess, paronychia, a boil, impetigo, an infection between the digits, anthrax, cellulitis, a secondary wound infection, otitis, sinusitis, hidradenitis, infectious mastitis, a post-traumatic skin infection or an infection of burnt skin.

16. A method for disinfecting or for preventing bacterial colonization by Gram-positive bacteria of a substrate comprising bringing the substrate into contact with a composition as claimed in claim 1.

17. The composition as claimed in claim 1, wherein said alkyl ether or alkyl acetal radical is in the 2-O-, 3-O-, and/or 4-O-position.

18. The composition as claimed in claim 2, wherein the deoxyhexose is rhamnose or fucose.

19. The composition as claimed in claim 4, wherein the deoxyhexose derivative is a methyl rhamnopyranoside

20. The composition as claimed in claim 7, wherein the Gram-positive bacteria are bacteria of the class Bacilli.
